(19) United States
(12) Patent Application Publication
(10) Pub. No.: US 2020/0016005 A1
(43) Pub. Date: Jan. 16, 2020

(54) EAR PROTECTION DEVICE

(71) Applicant: FLARE AUDIO TECHNOLOGIES LIMITED, Lancing (GB)

(72) Inventor: Davies Richard ROBERTS, Shoreham-by-Sea (GB)

(21) Appl. No.: 16/304,105

(22) PCT Filed: May 15, 2017
(86) PCT No.: PCT/GB2017/051351

§ 371 Date: Nov. 21, 2018

(30) Foreign Application Priority Data
May 26, 2016 (GB) 1609296.7
Sep. 22, 2016 (GB) 1616129.1

(51) Int. Cl. A61F 11/12; A61F 11/08

(52) U.S. Cl. CPC A61F 11/12; A61F 11/14; A61F 2011/085; A61F 11/08

(57) ABSTRACT

An ear protection device (10) to fit in a user's ear comprises an inner core (20) of non-compressible rigid material which is a metal or ceramic, and a compressible and resilient suspension layer (40), covering at least part of the inner core (20), for suspending and cushioning said device in a user's ear. The suspension layer (40) may comprise memory foam. The materials of the inner core (20), and of the suspension layer (40) differ very significantly in their characteristic impedances, so sound transmission from one material to the other is inhibited, and sound propagation through the ear canal is very largely prevented.

This invention relates to an ear protection device, such as an earplug that is placed by a user in their ear to reduce noise and prevent hearing damage.

An earplug is a device that is meant to be inserted in the ear canal to protect the user's ears from bud noises or the intrusion of water, foreign bodies, dust or excessive wind. Presently, there are several different types of earplugs for hearing protection:
1) Foam earplugs, usually made from either polyvinyl chloride (PVC) or polyurethane (PU) memory foam, which are compressed (rolled) and put into the ear canal, where they expand to plug it.
2) Silicone earplugs, which are rolled into a ball and carefully moulded to fit over the external portion of the ear canal.
3) Flanged earplugs, including most types of musicians' or ‘Wi-Fi’ earplugs. Musicians' earplugs are designed to attenuate sounds evenly across the audio band and thus minimise their effect on the user's perception of bass and treble levels. These are commonly used by musicians and technicians, both in the studio and in concert, to avoid overexposure to high volume levels. These are typically made of silicone. 4) Custom moulded earplugs, made from a mould of the wearer's ear and designed to precisely fit all ear canal shapes. Custom moulded is further divided into laboratory-made and “formed in place”. Custom shaped plugs are recommended for long-term use, since they are more comfortable and gentle to the skin and won't go too far into the ear canal.

It has been known for many years that there is a problem with the noise reduction capability of existing earplugs and ear defenders, as they cannot reduce sound well enough to comply with current health and safety laws on noise exposure.

The safe limit of sound is just 85 dB for prolonged exposure, and the acceptable time period decreases greatly as the sound level increases. Traditional earplugs and defenders are made from plastic, foam, rubber and silicone, and these materials cannot limit sound transfer very well, in particular because they do not adequately block lower frequencies. For example with sharp loud sounds such as may occur in loud music or gunshots, ear plugs made from these materials are not very successful at protecting the wearer from the noise.

Sound exposure is becoming a real issue for employers and governments across the world as there is no available way to protect people from noise that exceeds circa 115 dB. Currently available devices simply cannot achieve the silencing level that is required by health and safety laws. There have been attempts to combat this problem, such as by use of noise cancelling ear protection devices, but these are expensive when compared to simple plugs.

According to the present invention there is provided an ear protection device to fit to a user's ear comprising: a solid inner core of non-compressible rigid and dense material which is a metal or a ceramic; and a compressible and resilient suspension layer, covering at least part of the inner core, for suspending and cushioning said device in a user's ear.

The inner core of the device may be made of metal, such as stainless steel, mild steel, brass, titanium or aluminium, or of a ceramic material such as alumina, tungsten carbide or titanium carbide. Typically the material of the inner core is of density more than 2000 kg/m³, for example between 2500 kg/m³ and 10,000 kg/m³. For example aluminium is of density 2700 kg/m³, titanium is of density about 4500 kg/m³, steel is about 7800 kg/m³, and brass is of density about 8400 kg/m³; ceramics are typically of density between 2200 kg/m³, and 4000 kg/m³. The Young's modulus of elasticity is typically between 70 GPa and 350 GPa, for example aluminium has a modulus of 70 GPa, brass about 100 GPa, titanium about 115 GPa, and steel about 200 GPa; ceramics may have values in a similar range, for example fused silica may have a bulk modulus of 70 GPa, but for example alumina can have a Young's modulus of between 300 GPa and 380 GPa, and sialon (silicon nitride and aluminium oxide) can have a Young's modulus of about 280 GPa. There are no holes through the inner core, so the inner core is not tubular, and is not hollow.

The suspension layer is made of one or more of foam, memory foam, rubber or silicone. Such materials have a lower density, typically less than 1200 kg/m³, and a Young's modulus far less. For example a silicone rubber may have a density in the range 1100-1200 kg/m³ and a Young's modulus in the range 4 MPa to 12 MPa, while a memory foam or foam will have lower values for both density and Young's modulus. For example the density of a polyurethane foam may be between 10 and 300 kg/m³.

It will be appreciated that the materials of the inner core and of the suspension layer differ as regards their characteristic impedance, which is the product of density and the speed of sound, the speed of sound depending on the modulus of elasticity of the material and its density. By ensuring the material of the inner core and the material of the suspension layer differ very significantly in both density and modulus of elasticity, and therefore in their characteristic impedances, sound transmission from one material to the other is inhibited.

The inner core may be substantially cylindrical. It may be completely enclosed and embedded within the suspension layer. In this case the ear protection device would resemble a conventional ear plug, as the inner core would not be visible, and the ear protection device could be inserted into a user's ear canal and subsequently removed from it in the same way as a conventional ear plug.

In an alternative, the inner core may comprise a first substantially cylindrical portion, and a second substantially cylindrical portion of smaller diameter extending coaxially from an end of said first portion, and the second portion is enclosed and embedded within the suspension layer, or is surrounded by a tubular suspension layer. In this case the first cylindrical portion would be visible to the user, and the device would be inserted into the user's ear canal such that the suspension layer and the second portion are within the ear canal. The second substantially cylindrical portion may define one or more grooves, which may have angular edges; such grooves may enhance grip of the suspension layer to the second portion.

The ear protection device may also define a grippable portion at or near an end of the first portion opposite to that from which the second portion extends, so it is easier for the user to hold and remove the ear protection device from his ear canal. This grippable portion may be a projecting handle, for example a disc-like flange, or a groove around the periphery of the first portion.

Thus in use of one type of ear protection device at least part of the inner core is surrounded with the suspension layer and is located within the ear canal of the user so as to seal to the wall of the ear canal. In some cases the entire inner core locates within the ear canal in this way. Alternatively, part of the ear protection device may project outside the open end of the ear canal, and those parts of the ear protection device that are not intended to be inserted within the ear canal do not have to be surrounded by the suspension layer. They may therefore be decorative, for example being coated with an attractive metal such as gold or silver, or may be provided with a coloured finish by enamelling or (in the case of titanium for example) by anodising.

An alternative type of ear protection device fits at least partly into or against the user's outer ear. In this case the inner core of rigid material may for example be a flat plate, and the compressible and resilient suspension layer covers at least the face of the inner core closer to the user's head, providing a seal to the user's outer ear. The face of the inner core further from the user's head may be exposed, or may be covered for example by a layer of foam or other padding. This type of ear protection device is the equivalent of an ear defender, for which typically a pair are worn over both ears and resemble headphones.

Thus the invention is applicable to two different types of ear protection device. Where the ear protection device is designed to be inserted into the user's ear canal as an ear plug, then the suspension layer is arranged to seal to a wall of the ear canal. Alternatively, if the ear protection device is designed to cover the ear, then the suspension layer would be arranged to seal to the outer ear.

In each such ear protection device it is preferable if any corner of the inner core that may be expected to come into proximity with the skin of the user's ear or ear canal is rounded, to reduce the risk of the ear protection device scratching or abrading the skin. As regards those parts of the ear protection device that are expected to come into contact with the skin of the user's ear or ear canal, preferably there is at least 2 mm thickness of a cushioning resilient material between the outer surface of the ear protection device and any portion of the rigid inner core, more preferably at least 3 mm, when the cushioning resilient material is in its initial un-squeezed state; and preferably there is at least 0.5 mm thickness of the cushioning resilient material when the ear protection device is in situ within the ear, for example 1 mm or 1.5 mm. When the ear protection device is in situ, for example within an ear canal, along at least one path around the periphery of the ear protection device the cushioning resilient material is squeezed to less than its initial state, which ensures a seal between the ear protection device and the inner surface of the ear canal along that peripheral path.

The invention will now be further and more particularly described, by way of example only, and with reference to the accompanying drawings, in which:

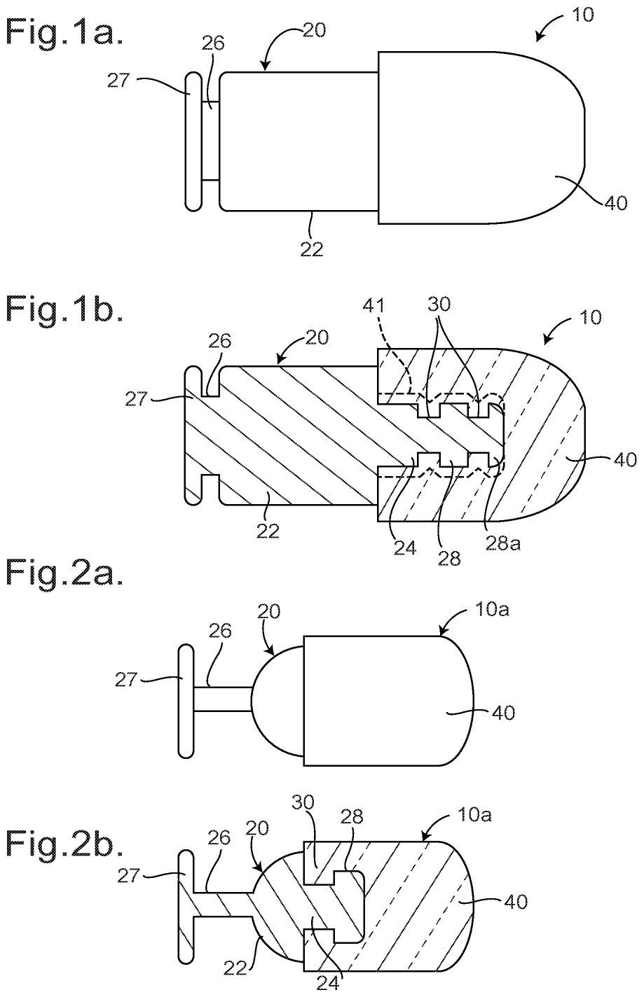

FIG. 1a shows a side view of an ear protection device;

FIG. 1b is a cross-sectional view of the ear protection device of FIG. 1a;

FIG. 2a shows a side view of an alternative ear protection device;

FIG. 2b is a cross-sectional view of the ear protection device of FIG. 2a;

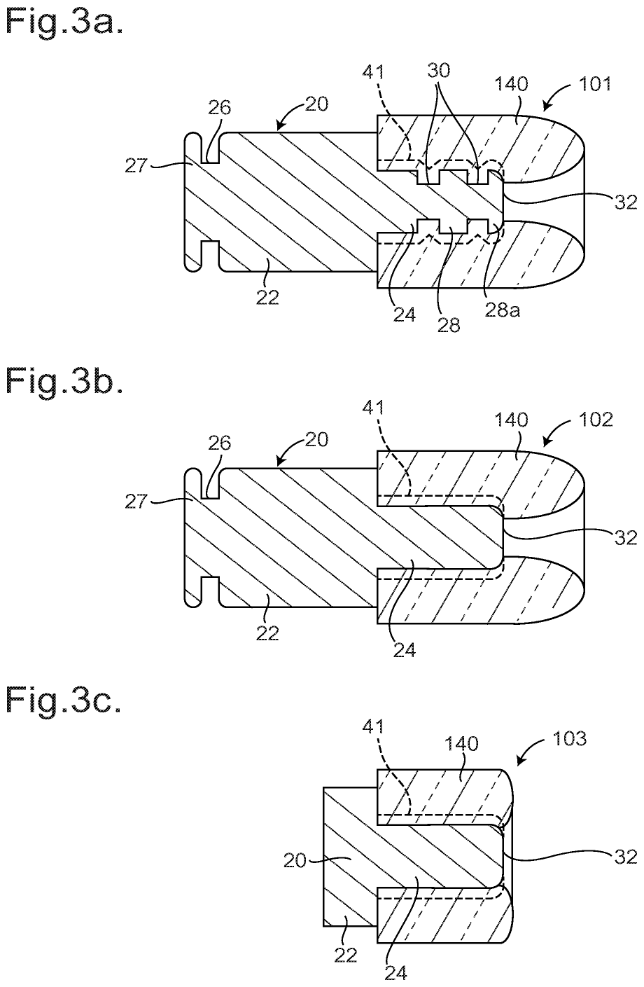

FIGS. 3a to 3c show cross-sectional views of modifications to the device of FIG. 1b;

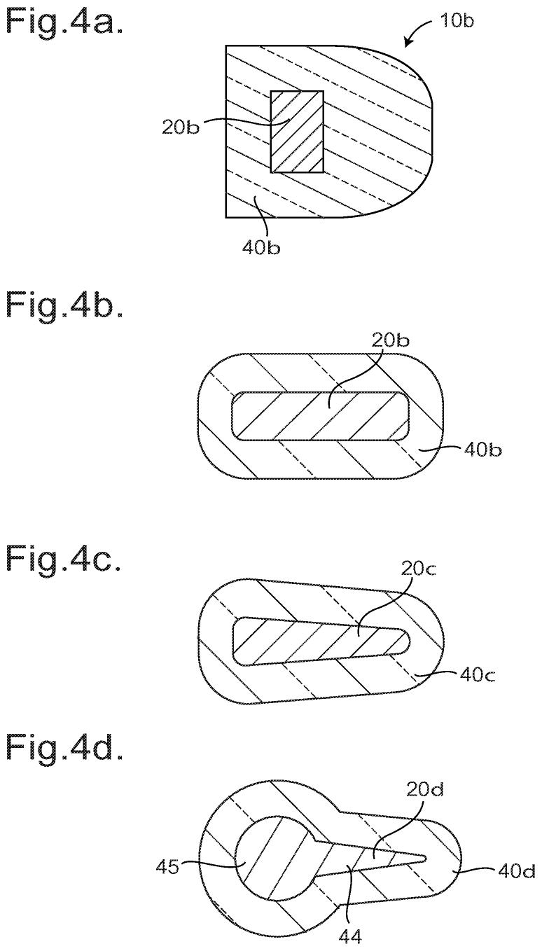

FIG. 4a shows a cross-sectional view of another alternative ear protection device;

FIGS. 4b to 4d show cross-sectional views of modifications to the device of FIG. 4a;

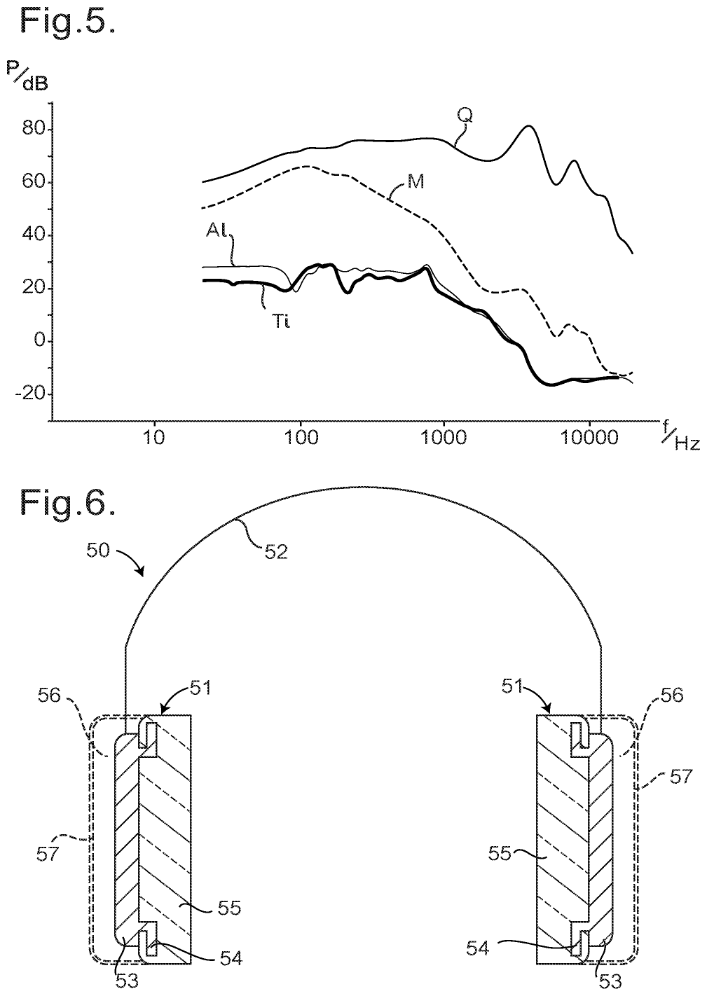

FIG. 5 shows graphically experimental data on attenuation obtained using devices of the invention; and

FIG. 6 shows a cross-sectional view of an alternative ear protection device.

FIGS. 1(a) and 1(b) show an ear protection device 10 which consists of a solid inner core 20, and a suspension cap 40. In this embodiment the inner core is 17.5 mm in length, but more generally it may be between 10 mm and 25 mm long; in this embodiment the diameter of the largest portion of the inner core 20 is 8.5 mm, but more generally this diameter may be between 5 mm and 10 mm. The inner core 20 includes a first cylindrical portion 22 that will protrude out of the ear canal when the device is inserted into the ear canal, and is the portion of largest diameter of the inner core 20. The first portion 22 also defines a circumferential notch or groove 26 to form a grippable end portion 27. The length of the first portion 22 including the end portion 27 is 11.5 mm in this case, but it more generally may be between 8 mm and 15 mm. In this embodiment, and as shown, the edges of the groove 26 may be curved, so that fingertips or fingernails can easily grip the end portion 27 to insert or remove the device 10 from a user's ear canal.

The solid inner core 20 is made of a non-compressible rigid and dense material, for example a metal such as steel, titanium or aluminium, or any other suitable metal, or from a ceramic material. The suspension cap 40 is made of a compressible material such as foam, memory foam, silicone or rubber; such a material has a significantly lower density and a much lower modulus of elasticity than the material of the solid core 20. In some cases the suspension cap 40 may be custom-moulded to an individual user's ears, and may be made from silicone or acrylic. The provision of the solid non-compressible inner core 20 inside the suspension cap 40 significantly enhances the silencing attribute of the device 10.

Inside the cap 40, the inner core 20 defines a second portion 24 of smaller diameter that extends into the cap 40. The diameter of the second portion 24 in this embodiment is 4 mm, but may be between 3 and 6 mm for example. The second portion 24 is provided with two circumferential grooves 30, so as to define a ridge 28 between the grooves 30 and an end ridge 28a between one of the grooves 30 and the end of the second portion 24. In this embodiment the grooves 30 are approximately 0.5 mm deep. The grooves 30 are not rounded, so they have angular edges.

As shown, the inner core 20 defines two grooves 30 on the second portion 24, but there may be a different number of grooves 30, or indeed no grooves 30. Where there are multiple grooves 30, these grooves 30 may all be the same width and depth, but in some embodiments a device may be provided with ridges 28 of different sizes (diameter and/or length), for example they may get smaller or larger the further away they are from the first portion 22 of the inner core 20, or they may be unevenly spaced along the second portion 24 of the inner core 20. The more ridges 28 that are provided on the inner core 24, the greater the sound attenuation is, so a particular material can be selected for the inner core 22 according to the use or application of the ear protector, as well as the overall price.

The suspension cap 40 is sized to be a snug fit to the inner core 20, and the opening of the cap 40 will fit up against the base of the first portion 22 of the inner core 20. All of the second portion 24 of the inner core 20 is therefore contained within the cap 40, and so is not visible. The suspension cap 40 is the portion of the device 10 that will be inserted into a user's ear.

The suspension cap 40 may for example be entirely of a material such as memory foam. Alternatively, as indicated by broken lines in FIG. 1b, the suspension cap 40 may consist of an inner tubular sleeve 41 of elastic material embedded within a material such as a memory foam, the tubular sleeve 41 being for example of a silicone elastomer and being a tight fit around the second portion 24 of the inner core 20, in this example the tubular sleeve 41 deforming into the grooves 30. The tubular sleeve 41 and its deformation into the grooves 30 ensure that the suspension cap 40 is securely attached to the inner core 20 during insertion and removal, and in use the suspension cap 40 supports and locates the inner core 20 so the second portion 24 is within the ear canal.

As with known ear plugs that include a memory foam, in its initial, un-squeezed state the external diameter of the suspension cap 40 is larger than that of the ear canal, but the suspension cap 40 can be squeezed or rolled between the user's fingers before being inserted into the ear canal, so that it goes in without difficulty; the memory foam then gradually expands to seal against the adjacent walls of the ear canal. The external diameter of the first portion 22 is less than that of the ear canal, so the inner core 22 does not come into contact with the ear canal.

FIGS. 2(a) and 2(b) show an alternative ear protector device 10a. Equivalent elements have been given the same reference numerals as used in FIGS. 1(a) and 1(b). In this device 10a, the second portion 24 of the inner core 22 only has a single ridge 28 and a single groove 30, and the outer face of the first portion 22 of the inner core 20 is slightly curved. In this device 10a the second cylindrical portion 24 does not extend so far into the wearer's ear canal.

The exact design of the device 10 or 10a can be balanced between comfort, simple insertion/removal and sound attenuation required by the user. The devices 10 and 10a feel extremely comfortable to use (sleeping overnight with them is possible, and the device is totally silent), as it is only the outer cap 40 that is in contact with the ear canal, so it is comfortable to wear. Careful design of the device 10 or 10a means that the inner core 20 need never touch the user's skin. The inner core 20 therefore may be described as acting as a floating isolator. In use, the exterior of the first cylindrical portion 22 of inner core 20 will sit just on the outside of the inner ear canal to block sound from reaching the user's ear.

In some embodiments of the invention the suspension cap 40 is disposable and can be replaced when required, whereas the inner core 20 is repeatedly re-usable. This would be the case for example for the devices 10 and 10a.

In some embodiments of the invention the suspension layer is tubular. FIGS. 3a, 3b and 3c show ear protection devices 101, 102 and 103 respectively which are modifications to the ear protection device 10; those features which are the equivalent of those in the ear protection device 10 are referred to by the same reference numerals.

Referring now to FIG. 3a, the ear protection device 101 differs from the ear protection device 10 in having a tubular suspension layer 140, so that the end 32 of the second portion 24 of the solid core 20, which is the end that is inserted into the ear, is exposed. In this example the tubular suspension layer 140 extends beyond the exposed end 32 of the solid core 20, and has a rounded shape so as to be comfortable when inserted into the ear.

Referring now to FIG. 3b, the ear protection device 102 differs from the ear protection device 101 in that the second portion 24 of the solid core 20 is cylindrical rather than grooved; the end 32 which is inserted into the ear has a rounded edge, as in the ear protection device 10.

Referring now to FIG. 3c, the ear protection device 103 differs from the ear protection device 102 in that the solid core 20 is shorter: the first cylindrical portion 22 that will protrude out of the ear canal when the device 103 is inserted into the ear canal, and which is the portion of largest diameter of the inner core 20, is shorter than in the ear protection device 102, and is not provided with the grippable end portion 27. The ear protection device 103 also differs from the ear protection device 102 in that the tubular suspension layer 140 is shorter and substantially cylindrical, so it does not project far beyond the end 32 of the second portion 24.

As another alternative, the ear protection device 103 might instead have a solid core 20 whose second cylindrical portion 24 defines one or more grooves 30, as in the ear protection devices 101 and 10. The provision of such grooves 30 enhances the grip between the inner tubular sleeve 41 of elastomeric material and the solid core 20.

In each of the ear protection devices 101, 102 and 103, as with the previously-described devices 10, the solid core 20 is made of a non-compressible rigid and dense material, for example a metal such as steel, titanium or aluminium, or any other suitable metal, or from a ceramic material, or any other rigid material of suitable density. The tubular suspension layer 140 is made of a compressible material such as foam, memory foam, silicone or rubber; such a material has a significantly lower density and a much lower modulus of elasticity than the material of the solid core 20.

In each of the ear protection devices 101, 102 and 103 the tubular suspension layer 140 may be disposable and can be replaced when required, whereas the inner core 20 is repeatedly re-usable.

It will be appreciated that a number of other modifications may be made, for example the ear protection device 101 or 102 might instead be provided with the shorter tubular suspension layer 140 as shown in FIG. 3c. Furthermore each of the ear protection devices 101 and 102 might instead have a shorter inner core 20 as shown in FIG. 2b.

In the devices 10 and 10a described above the first portion 22 of the inner core 20 is not covered by the suspension cap 40. In some alternatives, the inner core may be completely encapsulated by the suspension cap 40, and then the device may have the outward appearance of a traditional foam earplug. In this case, the suspension cap 40 may be formed as a single unit, with a slot through which the inner core can be inserted; or alternatively the suspension cap 40 may be formed by moulding around the inner core 20. The cap 40 is designed so that it forms an outer barrier between the inner core 20 and the wearer's ear, and can be compressed when the device is inserted into an ear. So for example, referring now to FIG. 3a, a cheaper and simpler disposable ear protection device 10b consists only of an inner core 20b that is of cylindrical shape and is entirely enclosed and embedded within an outer covering 40b that may comprise memory foam. In its external appearance the device 10b may therefore look the same as a conventional foam earplug device.

It will be appreciated that a modification of the device 10b may have a different external appearance from a conventional foam earplug. By way of example, as shown in FIG. 4b the inner core 20b might be cylindrical, of external diameter 4 mm and of length 15 mm, with rounded corners; and the outer covering 40b, in its initial un-squeezed state, may be of thickness about 3 mm on all surfaces, also having rounded corners. This device could be inserted into the user's ear canal in either length-wise direction.

In a further modification of the device 10b, as shown in FIG. 4c an inner core 20c may be tapered along its length, so having a part-conical shape, and in this case too the outer covering 40c in its initial un-squeezed state is of thickness about 3 mm on all surfaces, having rounded corners. The angle of taper of the inner core 20c and of the outer covering 40c should be substantially the same as the angle of taper of the ear canal.

In another modification of the device 10b, as shown in FIG. 4d an inner core 20d consists of a tapered portion 44 and also a spherical or bulbous portion 45 of larger diameter, and in this case also the outer covering 40d in its initial un-squeezed state is of thickness about 3 mm on all surfaces, having rounded corners.

Initial testing has shown that the ear protection devices of the invention achieve around −60 dB or more attenuation over all frequencies (broadband attenuation). When a user is wearing ear protection devices of the invention, normal conversation is still possible, whereas very loud sounds never appear loud to the user, even at very high extremes of 135 dB. When the device is inserted into a user's ears the user is able to have a normal conversion due to head/bone conduction of sound waves; but as the user is exposed to increasingly higher level sound the protected ears are never in danger of damage to the eardrum, as these intense sound waves are no longer propagating along the ear canal and so no longer have the normal typical push/pull effect on the eardrum. Over-stressing of the eardrum or damage to the cochlea is therefore prevented.

Referring now to FIG. 5, this shows graphically the variation in attenuation achieved experimentally with the earplug device 10 of FIG. 1, the graphs showing the variation in sound intensity, P, in decibels and its variation with frequency, f, in hertz. The measurements were carried out using a test microphone installed within a short thick-walled titanium tube, with an ear-canal sized aperture at the other end of the tube, the titanium tube and test microphone being mounted on a stand. The end of the tube with the aperture is within an acoustic box which also contains a small loudspeaker, and the microphone is outside the box. The loudspeaker is provided with a signal which gradually varies over the frequency range shown (from 20 Hz up to 20 kHz), and measurements are made with and without an earplug device 10 in the aperture, and also with a conventional foam earplug.

The line marked Q shows the reference signal without the use of the earplug device 10; the line marked M shows the variation of signal intensity when using a conventional foam earplug; the line marked Al shows the measured sound intensity in the case where the solid core 20 is of aluminium; while the line marked Ti is the measured sound intensity in the case where the solid core 20 is of titanium. At any particular frequency the attenuation achieved by a particular earplug device 10 is therefore given by the difference between the line Q and the line Al or Ti corresponding to the earplug device 10 in question.

It will be observed that the foam earplug, as shown by the line M, reduces the sound intensity over the entire frequency range, as is its purpose, but that for most frequencies the attenuation is markedly greater when using the earplug device 10.

It will be observed that the device 10 with the aluminium solid core 20, i.e. the line Al, gives an attenuation of about 40-50 dB in the vocal range; the attenuation gets gradually greater as the frequency increases, especially between about 800 Hz and 5 kHz. Although there is some fluctuation with frequency, there are no resonances. For frequencies up to over 10 kHz the attenuation is significantly greater than with the foam earplug.

Similarly it will be seen that the device 10 with the titanium solid core 20, i.e. the line Ti, performs similarly, the attenuation generally becoming greater as the frequency increases, but that at least for frequencies between about 20 Hz and 90 Hz the titanium gives about 7 dB more attenuation than was achieved by the device 10 with the aluminium solid core 20. Over the entire frequency range up to above 10 kHz the device 10 with the titanium solid core 20 gives considerably more attenuation than the foam earplug.

The ear protection devices described above are all intended to seal at least partly into the ear canal of the user, equivalent to a foam earplug. An alternative type of ear protection device seals to the outer ear, acting as an ear defender that resembles one of a pair of headphones. Referring now to FIG. 6, there is shown a sectional view of a pair 50 of ear defenders 51, linked by a resilient headband 52 to fit over the user's head so that the ear defenders 51 are up against the user's ears.

Each ear defender 51 consists of a flat circular plate 53 of rigid material with a stepped projecting flange 54 around its perimeter, and a circular layer 55 of resilient compressible material that connects to the projecting flange 54 and seals to the user's outer ear.

The plate 53 provides the inner core of the device, and may be made of metal, such as stainless steel, mild steel, brass, titanium or aluminium, or of a ceramic material such as alumina, tungsten carbide or titanium carbide. It may have a different shape to that illustrated. The layer 55 corresponds to the suspension layer, and may be made of one or more of foam, memory foam, rubber or silicone. By way of modifications, as indicated in broken lines, the outside face of each rigid plate 53, i.e. the face further from the ear, is covered with a further layer 56 of resilient compressible material. This external layer 56, which may for example be of foam or memory foam, rubber or silicone, may also be enclosed within a thin casing 57 for example of moulded plastic.

Other variations and modifications will be apparent to the skilled person. Such variations and modifications may involve equivalent and other features that are already known and which may be used instead of, or in addition to, features described herein. Features that are described in the context of separate embodiments may be provided in combination in a single embodiment. Conversely, features that are described in the context of a single embodiment may also be provided separately or in any suitable sub-combination.

It should be noted that the term “comprising” does not exclude other elements or steps, the term “a” or “an” does not exclude a plurality, a single feature may fulfil the functions of several features recited in the claims and reference signs in the claims shall not be construed as limiting the scope of the claims. It should also be noted that the Figures are not necessarily to scale; emphasis instead generally being placed upon illustrating the principles of the present invention.

1. An ear protection device to fit to a user's ear comprising:
an inner core of non-compressible rigid and dense material which is a metal or a ceramic, the inner core being solid so it is not tubular and is not hollow; and
a compressible and resilient suspension layer, covering at least part of the inner core, for suspending and cushioning said device in or to a user's ear;
wherein the inner core comprises at least a portion engaging with the outer suspension layer, and the engaging portion defines one or more circumferential grooves.

2. An ear protection device as claimed in claim 1 wherein the inner core of the device is of metal, and the metal is steel, titanium or aluminium.

3. An ear protection device as claimed in claim 1 wherein the compressible and resilient suspension layer comprises one or more of foam, memory foam, rubber or silicone.

4. An ear protection device as claimed in claim 1 wherein the inner core is completely enclosed and embedded within the suspension layer.

5. An ear protection device as claimed in claim 4 wherein the inner core is substantially cylindrical.

6. An ear protection device as claimed in claim 1 adapted to fit at least partly into or against the user's outer ear, wherein the compressible and resilient suspension layer covers at least a face of the inner core closer to the user's head, providing a seal to the user's outer ear.

7. An ear protection device as claimed in claim 6 wherein the inner core is generally plate-shaped, being spaced apart from the ear by the compressible and resilient suspension layer.

8. An ear protection device as claimed in claim 6 wherein the surface of the inner core further from the user's head is covered with foam or padding.

9. An ear protection device as claimed in claim 4 wherein the inner core is tapered along at least part of its length.

10. An ear protection device as claimed in claim 4 wherein the inner core includes a part-spherical portion and a tapered portion.

11. An ear protection device as claimed in claim 1 wherein the inner core comprises a first substantially cylindrical portion, and a second substantially cylindrical portion of smaller diameter extending coaxially from an end of said first portion, and the second portion is the engaging portion, and defines the one or more circumferential grooves.

12-13. (canceled)

14. An ear protection device as claimed in claim 1 wherein the inner core defines a first portion which extends from an end of the engaging portion, and also defines a grippable portion at or near an end of the first portion opposite to that from which the engaging portion extends.

15. An ear protection device as claimed in claim 1 wherein any corner of the inner core that may be expected to come into proximity with the skin of the user's ear or ear canal is rounded, to reduce the risk of the ear protection device scratching or abrading the skin.

16. An ear protection device as claimed in claim 1 wherein those parts of the ear protection device that are expected in use to come into contact with the skin of the user's ear or ear canal, include at least 2 mm thickness of the compressible and resilient suspension layer between the outer surface of the ear protection device and any portion of the rigid inner core, more preferably at least 3 mm, when the suspension layer is in its initial un-squeezed state.

17. An ear protection device as claimed in claim 7 wherein the inner core is generally plate-shaped, and wherein the circumferential groove is defined by a stepped projecting flange around the perimeter of the plate.
